(19) United States
(12) Patent Application Publication
(10) Pub. No.: US 2023/0240594 A1
(43) Pub. Date: Aug. 3, 2023

(54) POSTURE ASSESSMENT PROGRAM, POSTURE ASSESSMENT APPARATUS, POSTURE ASSESSMENT METHOD, AND POSTURE ASSESSMENT SYSTEM

(71) Applicants: Rika TAKAGI, Aichi (JP); Kousuke ARIGA, Tokyo (JP)

(72) Inventor: Kousuke ARIGA, Tokyo (JP)

(73) Assignees: Rika TAKAGI, Aichi (JP); Kousuke ARIGA, Tokyo (JP)

(21) Appl. No.: 18/015,618

(22) PCT Filed: Jun. 18, 2021
(86) PCT No.: PCT/JP2021/023272

§ 371 Date: Jan. 11, 2023

(30) Foreign Application Priority Data
Sep. 9, 2020 (JP) 2020-151655

(51) Int. Cl. A61B 5/00; G06T 7/73; G06T 15/00; A63B 71/06

(52) U.S. Cl. CPC A61B 5/4561; G06T 7/74; G06T 15/005; A63B 71/0622; A63B 2071/0636; A63B 2220/05

(57) ABSTRACT

The object of the present invention is to provide a posture assessment program, a posture assessment apparatus, a posture assessment method, and a posture assessment system, which are capable of grasping a posture state.

A posture assessment program to be executed on a computer apparatus, the posture assessment program causing the computer apparatus to function as: a first identifier configured to identify at least two points of a body site of an assessed person; and an orientation identifier configured to identify an orientation of the body site, based on the points that have been identified by the first identifier.

### TECHNICAL FIELD

The present invention relates to a posture assessment program, a posture assessment apparatus, a posture assessment method, and a posture assessment system, which are capable of grasping a posture state of human body.

### BACKGROUND ART

In order for people to maintain their health conditions, it is important to do moderate exercise on a daily basis. In these years, in order to enhance physical functions, the number of people who do exercise by using training facilities or the like is also increasing. In addition, also in treatment facilities such osteopathic clinics, chiropractic clinics, or rehabilitation facilities, by making the patients do various exercises, a so-called exercise therapy is given to reduce symptoms, provide treatment, or restore functions of patients.

In order to enhance the effects of exercise, it is necessary to assess human body conditions carefully and determine an exercise menu so as to make them do exercise in a proper form. For example, even in a case where an exercise menu is generally encouraged, when the physical condition of a person is not in a condition suitable for doing such an exercise menu or a person is doing the menu in an improper form, although the exercise menu is done, muscles that are originally not to be used are used, or an excessive load is applied to joints and soft tissues around joints. This may lead to troubles or indefinite complaints.

In order to provide a more effective exercise menu that does not lead to troubles or unidentified complaints, it is preferable to quantitatively grasp the posture state of the person. In addition, in order to make the person do an exercise menu in a proper form, it is desirable to visualize differences between the form of the person and an ideal form and make the person who does the exercise recognize the differences. When it is possible to grasp these posture states, a more appropriate exercise menu for the person can be provided to make the person do exercise properly.

### SUMMARY OF INVENTION

### Technical Problem

The present invention has been made in view of the above circumstances. That is, the object of the present invention is to provide a posture assessment program, a posture assessment apparatus, a posture assessment method, and a posture assessment system, which are capable of grasping a posture state.

### Solution to Problem

The present invention is to solve the above problems by providing the following [1] to [27].

[1] A posture assessment program to be executed on a computer apparatus, the posture assessment program causing the computer apparatus to function as: a first identifier configured to identify at least two points of a body site of an assessed person; and an orientation identifier configured to identify an orientation of the body site, based on the points that have been identified by the first identifier;
[2] The posture assessment program according to [1], wherein the posture assessment program causes the computer apparatus to further function as: a second identifier configured to identify at least one point of the body site; and an orientation displayer configured to display information regarding the orientation that has been identified in association with the point that has been identified by the second identifier;
[3] The posture assessment program according to [1] or [2], wherein a plurality of the body sites are present, and the posture assessment program causes the computer apparatus to further function as a positional relationship displayer configured to display information indicating a positional relationship between a position of one body site and a position of another body site, based on the point that has been identified by the second identifier for each body site;
[4] A posture assessment apparatus comprising: a first identifier configured to identify at least two points of a body site of an assessed person; and an orientation identifier configured to identify an orientation of the body site, based on the points that have been identified by the first identifier;
[5] A posture assessment method to be performed on a computer apparatus, the posture assessment method comprising: a first identification step of identifying at least two points of a body site of an assessed person; and an orientation identification step of identifying an orientation of the body site, based on the points that have been identified by the first identification step;
[6] A posture assessment system comprising: a first apparatus; and a second apparatus capable of conducting a communication connection with the first apparatus; a first identifier configured to identify at least two points of a body site of an assessed person; and an orientation identifier configured to identify an orientation of the body site, based on the points that have been identified by the first identifier;
[7] A posture assessment method comprising: a first identification step of identifying at least two points of a body site of an assessed person; and an orientation identification step of identifying an orientation of the body site, based on the points that have been identified by the first identification step;
[8] A posture assessment program to be executed on a computer apparatus, the posture assessment program causing the computer apparatus to function as: a second identifier configured to identify at least one point for each body site of a plurality of body sites of an assessed person; and a positional relationship displayer configured to display information indicating a positional relationship between a position of one body site and a position of another body site, based on the point that has been identified by the second identifier for each body site;
[9] A posture assessment apparatus comprising: a second identifier configured to identify at least one point for each body site of a plurality of body sites of an assessed person; and a positional relationship displayer configured to display information indicating a positional relationship between a position of one body site and a position of another body site, based on the point that has been identified by the second identifier for each body site;
[10] A posture assessment method to be performed on a computer apparatus, the posture assessment method comprising: a second identification step of identifying at least one point for each body site of a plurality of body sites of an assessed person; and a positional relationship display step of displaying information indicating a positional relationship between a position of one body site and a position of another body site, based on the point that has been identified by the second identification step for each body site;
[11] A posture assessment system comprising: a first apparatus; and a second apparatus capable of conducting a communication connection with the first apparatus; a second identifier configured to identify at least one point for each body site of a plurality of body sites of an assessed person; and a positional relationship displayer configured to display information indicating a positional relationship between a position of one body site and a position of another body site, based on the point that has been identified by the second identifier for each body site;
[12] A posture assessment method comprising: a second identification step of identifying at least one point for each body site of a plurality of body sites of an assessed person; and a positional relationship display step of displaying information indicating a positional relationship between a position of one body site and a position of another body site, based on the point that has been identified by the second identification step for each body site;
[13] A posture assessment program to be executed on a computer apparatus, the posture assessment program causing the computer apparatus to function as an orientation displayer configured to display information regarding an orientation to be identified according to a sensor attached to at least one point of a body site of an assessed person, as information regarding the orientation of the body site;
[14] A posture assessment apparatus comprising an orientation displayer configured to display information regarding an orientation to be identified according to a sensor attached to at least one point of a body site of an assessed person, as information regarding the orientation of the body site;
[15] A posture assessment method to be performed on a computer apparatus, the posture assessment method comprising an orientation display step of displaying information regarding an orientation to be identified according to a sensor attached to at least one point of a body site of an assessed person, as information regarding the orientation of the body site;
[16] A posture assessment system comprising: a first apparatus; and a second apparatus capable of conducting a communication connection with the first apparatus; and an orientation displayer configured to display information regarding an orientation to be identified according to a sensor attached to at least one point of a body site of an assessed person, as information regarding the orientation of the body site;
[17] A posture assessment system comprising: a sensor attached to at least one point of a body site of an assessed person; and a computer apparatus, wherein the computer apparatus includes an orientation displayer configured to display information regarding an orientation to be identified according to the sensor, as information regarding the orientation of the body site;
[18] A posture assessment program to be executed on a computer apparatus, the posture assessment program causing the computer apparatus to function as a positional relationship displayer configured to display information indicating a positional relationship between a position of one body site and a position of another body site, based on a position to be identified according to a sensor attached to at least one point of each of a plurality of body sites of an assessed person;
[19] A posture assessment apparatus comprising a positional relationship displayer configured to display information indicating a positional relationship between a position of one body site and a position of another body site, based on a position to be identified according to a sensor attached to at least one point of each of a plurality of body sites of an assessed person;
[20] A posture assessment method to be performed on a computer apparatus, the posture assessment method comprising a positional relationship display step of displaying information indicating a positional relationship between a position of one body site and a position of another body site, based on a position to be identified according to a sensor attached to at least one point of each of a plurality of body sites of an assessed person;
[21] A posture assessment system comprising: a first apparatus; and a second apparatus capable of conducting a communication connection with the first apparatus; and a positional relationship displayer configured to display information indicating a positional relationship between a position of one body site and a position of another body site, based on a position to be identified according to a sensor attached to at least one point of each of a plurality of body sites of an assessed person;
[22] A posture assessment system comprising: a sensor attached to at least one point of a body site of an assessed person; and a computer apparatus, wherein the computer apparatus includes a positional relationship displayer configured to display information indicating a positional relationship between a position of one body site and a position of another body site, based on a position to be identified according to a sensor attached to at least one point of each of a plurality of body sites of an assessed person;
[23] A posture assessment program to be executed on a computer apparatus, the posture assessment program causing the computer apparatus to function as: an orientation identifier configured to identify an orientation of at least one point of each of a plurality of body sites of an assessed person; a position identifier configured to identify the position of the at least one point of each of the plurality of body sites; a virtual skeleton change part configured to change a virtual skeleton set in a virtual model in accordance with the orientation that has been identified by the orientation identifier and/or the position that has been identified by the position identifier; and a virtual model displayer configured to render a virtual model in accordance with the virtual skeleton that has been changed, and configured to display the virtual model as a two-dimensional image or a three-dimensional image;
[24] The posture assessment program according to [23], wherein the posture assessment program causes a computer apparatus to further function as an operation performing part configured to cause a virtual model to perform a predetermined operation;
[25] A posture assessment apparatus comprising: an orientation identifier configured to identify an orientation of at least one point of each of a plurality of body sites of an assessed person; a position identifier configured to identify the position of the at least one point of each of the plurality of body sites; a virtual skeleton change part configured to change a virtual skeleton set in a virtual model in accordance with the orientation that has been identified by the orientation identifier and/or the position that has been identified by the position identifier; and a virtual model displayer configured to render a virtual model in accordance with the virtual skeleton that has been changed, and configured to display the virtual model as a two-dimensional image or a three-dimensional image;
[26] A posture assessment method to be performed on a computer apparatus, the posture assessment method comprising: an orientation identification step of identifying an orientation of at least one point of each of a plurality of body sites of an assessed person; a position identification step of identifying the position of the at least one point of each of the plurality of body sites; a virtual skeleton change step of changing a virtual skeleton set in a virtual model in accordance with the orientation that has been identified by the orientation identification step and/or the position that has been identified by the position identification step; and a virtual model display step of rendering a virtual model in accordance with the virtual skeleton that has been changed, and displaying the virtual model as a two-dimensional image or a three-dimensional image;
[27] A posture assessment system comprising: a first apparatus; and a second apparatus capable of conducting a communication connection with the first apparatus; an orientation identifier configured to identify an orientation of at least one point of each of a plurality of body sites of an assessed person; a position identifier configured to identify the position of the at least one point of each of the plurality of body sites; a virtual skeleton change part configured to change a virtual skeleton set in a virtual model in accordance with the orientation that has been identified by the orientation identifier and/or the position that has been identified by the position identifier; and a virtual model displayer configured to render a virtual model in accordance with the virtual skeleton that has been changed, and configured to display the virtual model as a two-dimensional image or a three-dimensional image.

### Advantageous Effects of Invention

According to the present invention, it is possible to provide a posture assessment program, a posture assessment apparatus, a posture assessment method, and a posture assessment system, which are capable of grasping a posture state.

### BRIEF DESCRIPTION OF THE DRAWINGS

FIG. 1 is a block diagram illustrating a configuration of a computer apparatus according to an embodiment of the present invention.

FIG. 2 is a flowchart illustrating the posture assessment process according to an embodiment of the present invention.

FIG. 3 is a diagram illustrating an example of a display screen according to an embodiment of the present invention.

FIG. 4 is a diagram illustrating an example of a display screen according to an embodiment of the present invention.

FIG. 5 is a diagram illustrating an example of a display screen according to an embodiment of the present invention.

FIG. 6 is a diagram illustrating an example of a display screen according to an embodiment of the present invention.

FIG. 7 is a diagram illustrating an exercise menu table according to an embodiment of the present invention.

FIG. 8 is a flowchart illustrating an avatar display process according to an embodiment of the present invention.

FIG. 9 is a diagram illustrating a virtual skeleton model according to an embodiment of the present invention.

FIG. 10 is a block diagram illustrating a configuration of a posture assessment system according to an embodiment of the present invention.

### DESCRIPTION OF EMBODIMENTS

According to the present invention, the posture state of a person can be grasped accurately in a simple method. In a case where the posture state of an assessed person can be grasped, the balance of muscles of the assessed person can be grasped, for example, the position of a muscle in a hypertonic (contracted) state (hereinafter, also referred to as “tension muscle”) or a muscle in a hypotonic (relaxed or attenuated) state (hereinafter, also referred to as “relaxed muscle”). In a case where the states of the muscles can be grasped, it becomes possible to provide an exercise menu that appropriately works on each muscle, that is, an exercise menu more appropriate for the assessed person, and to make the assessed person do the exercise menu properly.

Hereinafter, embodiments of the invention will be described with reference to the drawings and the like. The invention, however, is not limited to the following embodiments without departing from the spirit of the invention. Further, the order of respective processes that form a flowchart described below may be changed in a range without contradicting or creating discord with the processing contents thereof.

Note that, in the present specification, a term “client” refers to an assessed person whose posture is to be assessed, and includes, for example, a user of a training facility, a sports amateur, an athlete, a patient in an exercise therapy, and the like. In addition, a term “trainer” refers to a person who gives an exercise instruction or advice to the client, and includes, for example, an instructor in a training facility, a sports trainer, a coach, a judo healing practitioner, a physical therapist, and the like. Further, a term “image” may be either a still image or a moving image.

### First Embodiment

First, an outline of a first embodiment of the present invention will be described. Hereinafter, as the first embodiment, a description will be given, as an example, for a program for causing a computer apparatus to assess a state of a client's posture.

According to the program in the first embodiment, for example, an image of a client's posture is captured by the trainer or the client itself, so that the client's posture can be grasped, based on the image that has been captured. As a result, it becomes possible to provide an exercise menu appropriate for the client.

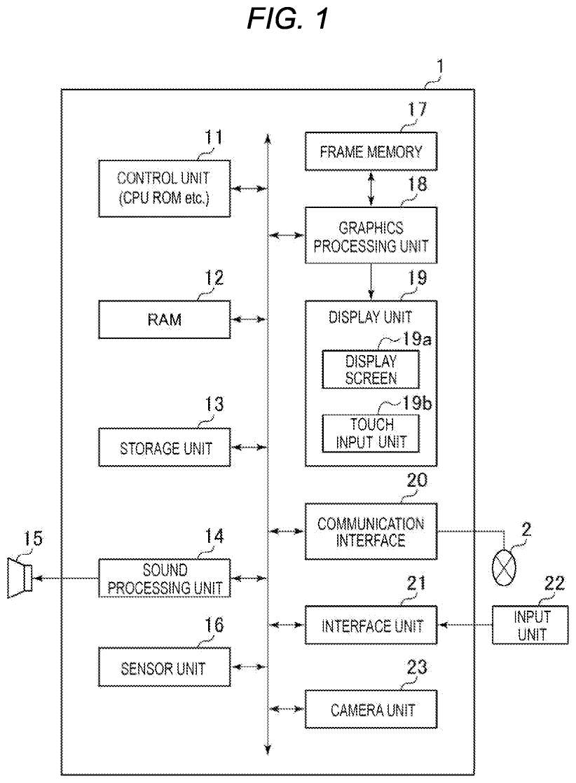

FIG. 1 is a block diagram illustrating a configuration of a computer apparatus according to an embodiment of the present invention. A computer apparatus 1 includes at least a control unit 11, a random access memory (RAM) 12, a storage unit 13, a sound processing unit 14, a sensor unit 16, a graphics processing unit 18, a display unit 19, a communication interface 20, an interface unit 21, and a camera unit 23, and they are respectively connected with one another through an internal bus.

The computer apparatus 1 is a terminal to be operated by a user (for example, a trainer or a client). Examples of the computer apparatus 1 include, but are not limited to, a personal computer, a smartphone, a tablet terminal, a mobile phone, a PDA, a server apparatus, and the like. The computer apparatus 1 is preferably communicably connectable with another computer apparatus through a communication network 2.

Examples of the communication network 2 include various known wired or wireless communication networks, such as the Internet, a wired or wireless public telephone network, a wired or wireless LAN, and a dedicated line.

The control unit 11 includes a CPU and a ROM, and includes an internal timer that counts time. The control unit 11 executes a program stored in the storage unit 13, and controls the computer apparatus 1. The RAM 12 is a work area of the control unit 11. The storage unit 13 is a storage area for storing programs and data.

The control unit 11 reads a program and data from the RAM 12, and performs a process. The control unit 11 processes the program and data that have been loaded onto the RAM 12, and outputs a sound output instruction to the sound processing unit 14 or outputs a drawing command to the graphics processing unit 18.

The sound processing unit 14 is connected with a sound output device 15, which is a speaker. When the control unit 11 outputs a sound output instruction to the sound processing unit 14, the sound processing unit 14 outputs a sound signal to the sound output device 15. The sound output device 15 is also capable of outputting, for example, an instruction regarding a client's posture and an exercise content, feedback on the exercise, and the like by sounds.

The sensor unit 16 includes at least one or more sensors selected from the group consisting of a depth sensor, an acceleration sensor, a gyro sensor, a GPS sensor, a fingerprint authentication sensor, a proximity sensor, a magnetic force sensor, a luminance sensor, a GPS sensor, and an atmospheric pressure sensor.

The graphics processing unit 18 is connected with the display unit 19. The display unit 19 includes a display screen 19a. In addition, the display unit 19 may include a touch input unit 19b. When the control unit 11 outputs a drawing command to the graphics processing unit 18, the graphics processing unit 18 expands an image in a frame memory 17, and outputs a video signal for displaying an image on the display screen 19a. The touch input unit 19b receives an operation input of the user, detects pressing by a finger, a stylus, or the like on the touch input unit 19b or a movement of the position of the finger or the like, and detects a change or the like in its coordinate position. The display screen 19a and the touch input unit 19b may be integrally configured to be like a touch panel, for example. The graphics processing unit 18 draws one image in units of frames.

The communication interface 20 is connectable with the communication network 2 wirelessly or by wire, and is capable of transmitting and receiving data through the communication network 2. The data that have been received through the communication network 2 are loaded onto the RAM 12, and an arithmetic process is performed by the control unit 11.

An input unit 22 (for example, a mouse, a keyboard, or the like) can be connected to the interface unit 21. Input information from the input unit 22 by the user is stored in the RAM 12, and the control unit 11 performs various arithmetic processes, based on the input information.

The camera unit 23 captures an image of the client, and images, for example, a client's posture in a stationary state and/or a moving state, a state in which the client is doing exercise, and the like. The image that has been captured by the camera unit 23 is output to the graphics processing unit 18. Note that the camera unit 23 does not have to be included in the computer apparatus 1, and, for example, may take in an image that has been captured by an external imaging device so as to acquire the image of the client that has been captured.

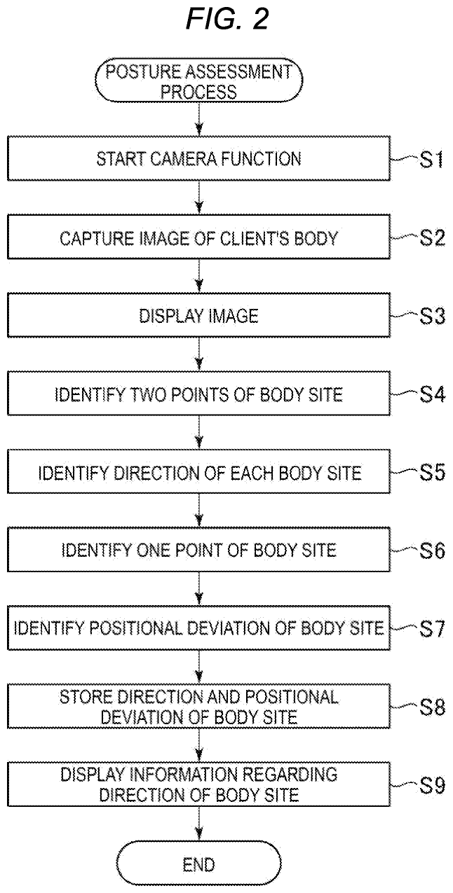

Next, a posture assessment process of the computer apparatus according to an embodiment of the present invention will be described. FIG. 2 is a flowchart illustrating the posture assessment process according to an embodiment of the present invention. When a user (for example, a trainer or a client) activates a dedicated application (hereinafter, a dedicated app) installed in the computer apparatus 1, and selects a start button of a camera function, the camera function is started (step S1). The user uses the computer apparatus 1 to capture an image of a part or an entirety of a client's body, for example, from a front direction or a lateral side direction (step S2). The image of the client is captured in a stationary state and in a state where the client lowers both arms and stands on both legs in a direction perpendicular to a horizontal plane. Note that it is also possible to capture an image of a part or an entirety of the client's body from a direction other than the front direction and the lateral side direction (for example, from a height direction). In addition, in order to more accurately grasp the posture state, preferably, the client wears clothes from which the body line can be recognized as much as possible.

Here, capturing an image from the front direction means capturing an image from a direction in which a person's face can be seen and a person's body can be visually recognized symmetrically. In addition, capturing an image in the lateral side direction means capturing an image from a direction perpendicular to the front direction and parallel to the horizontal plane, and means capturing an image from either a left or right direction of a human body. These images are preferably captured such that one side of the image is perpendicular or parallel to the horizontal plane. Note that capturing an image in the height direction means capturing an image from a direction perpendicular to the horizontal plane.

Note that, here, in step S2, the image of the client is captured with the camera function. However, image data of an image that has been captured by another computer apparatus or the like may be taken into the computer apparatus 1 to be used in step S3 and later steps. Further, in this case, the image may be not only a still image but also a moving image.

Next, the captured image of the client is displayed on the display screen 19a (step S3). The user visually recognizes the image of the client displayed on the display screen 19a, and identifies at least two points of a body site (step S4). Examples of a body site to be a point identification target in step S4 include a head part, a chest part, and a pelvis part, but another body site may be included as the point identification target. In step S4, at least two points are identified in each body site. These two points are used for identifying the orientation (inclination) of the body site, and which part of the body should be set as two predetermined points is preferably determined beforehand for each body site.

In addition, even in the same body site, the two predetermined points are preferably different between a case where the image has been captured from the front direction and a case where the image has been captured from the lateral side direction. In the case where the image has been captured from the front direction, it is possible to grasp the orientation in the height direction on the left and right of the body, and in the case where the image has been captured from the lateral side direction, it is possible to grasp the orientation in the height direction on the front side and rear side of the body.

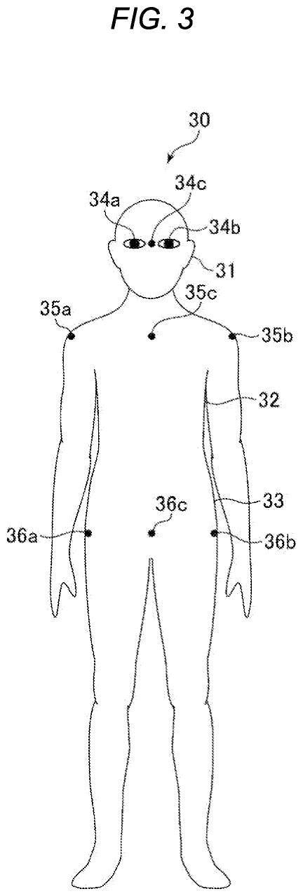

FIG. 3 is a diagram illustrating an example of a display screen according to an embodiment of the present invention. A captured image of a client's body from the front direction is displayed on the display screen. A body 30 includes a head part 31, a chest part 32, and a pelvis part 33. In the case of the image that has been captured from the front direction, for example, centers 34a and 34b of both eyes can be set as two predetermined points for the head part 31, acromioclavicular joints 35a and 35b (for example, a part corresponding to a connection part between the clavicle and the scapula, that is, a part assumed to be closest to the connection portion) of both shoulders can be set as two predetermined points for the chest part 32, and left and right anterior superior iliac spines 36a and 36b of the pelvis (a part assumed to be closest to a point protruding in a left-and-right direction of the pelvis) can be set as two predetermined points for the pelvis part 33.

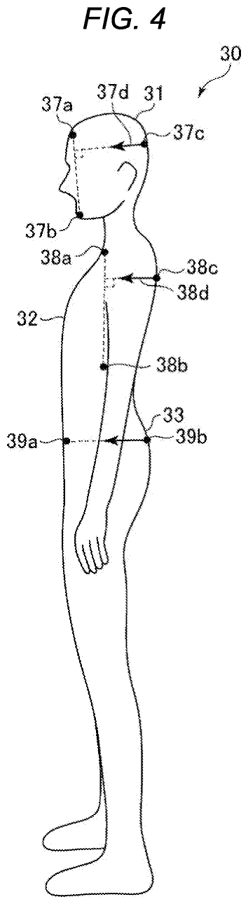

FIG. 4 is a diagram illustrating an example of a display screen according to an embodiment of the present invention. A captured image of a client's body 30 from the lateral side direction is displayed on the display screen. In the case of the image that has been captured from the lateral side direction, for example, a glabella 37a and a chin tip 37b can be set as two predetermined points for the head part 31, and a part 38a corresponding to the manubrium of sternum (a part assumed to be closest to the manubrium of sternum) and a part 38b corresponding to the tenth rib lower edge (a part assumed to be closest to the tenth rib lower edge) can be set as two predetermined points for the chest part 32. In addition, a part 39a corresponding to the anterior superior iliac spine (a part assumed to be closest to the ilium) and a second spinous process of vertebra 39b can be set as two predetermined points for the pelvis part 33.

In step S4, the two points for identifying the orientation of the body site may be identified in the image that has been captured from either one of the front direction or the lateral side direction. However, the two points may be identified in the image captured from a plurality of directions such as the front direction and the lateral side direction. By identifying totally four points for each body site in the front direction and the lateral side direction in this manner, it becomes possible to grasp the orientations in left-and-right direction and front-and-rear direction of each body site.

In identifying the point of the body site in step S4, it is possible to identify the point by performing a touch operation on the touch panel with a finger. However, for example, the point may be identified by performing a touch operation on the touch panel with a stylus, or the point may be identified by the user moving a cursor to a desired point on the image by operating the input unit 22. In addition, separately from the method for identifying points of the body site in accordance with an operation of the user, a method for automatically identifying the two predetermined points of the body site from the image data in accordance with a predetermined computer program or by a process performed by AI may be adopted.

Next, the orientation is identified for each body site, based on the two points that have been identified for each body site (step S5). For example, as in the case where acromioclavicular joints 35a and 35b of both shoulders are identified for the chest part, in a case where two points that are parallel to the horizontal plane are identified in a normal posture state, it is possible to represent the orientation of the body site with use of a line segment connecting the two points. In this case, in step S5, it is possible to identify the orientation of the body site with a parameter such as an angle formed by the line segment connecting the two points and a straight line perpendicular to the horizontal plane or a straight line parallel to the horizontal plane in the image, a vector starting from either one of the points and ending at the other, or the like.

Further, for example, as in the case where the glabella 37a and the chin tip 37b are identified for the head part, in a case where two points perpendicular to the horizontal plane are identified in the normal posture state, it is possible to represent the orientation of the body site with use of a normal line of the line segment connecting the two points. In this case, in step S5, it is possible to identify the orientation of the body site with a parameter such as an angle formed by the normal line of the line segment connecting two points and a straight line perpendicular to a horizontal plane or a straight line parallel to the horizontal plane in the image, and a normal line vector of the line segment connecting two points.

Next, the user visually recognizes the image of the client displayed on the display screen 19a, and identifies at least one point of the body site (step S6). Examples of the body site to be a point identification target in step S6 include the head part, the chest part, and the pelvis part, but another body site may be included as a point identification target. In step S6, at least one point is identified in each body site. Such one point is used for grasping a positional deviation of the body site, and which part of the body should be set as one predetermined point is preferably determined beforehand for each body site. In addition, the points identified in step S6 may include the point identified in step S4, that is, the points identified in step S4 and step S6 may be the same with each other, or may be different from each other.

Further, even in the same body site, one predetermined point is preferably different between a case where the image has been captured from the front direction and a case where the image has been captured from the lateral side direction. In the case where the image has been captured from the front direction, it is possible to grasp a deviation of the body site in the left-and-right direction. In the case where the image has been captured from the lateral side direction, it is possible to grasp a deviation of the body in the front-and-rear direction.

These predetermined points of the body site to be identified are preferably points aligned on a straight line, in a case of a person in a normal posture. In the image that has been captured from the front direction, the points to be identified in the head part, the chest part, and the pelvis part are preferably points aligned on a straight line, in a case of a person in a normal posture. Similarly, in the image that has been captured from the lateral side direction, the points to be identified in the head part, the chest part, and the pelvis part are preferably points aligned on a straight line, in a case of a person in a normal posture. With such a configuration, in a case where these points are not aligned on a straight line, it is possible to grasp that a deviation occurs in the position of the body site.

FIG. 3 is a diagram illustrating an example of a display screen according to an embodiment of the present invention. A captured image of a client's body from the front direction is displayed on the display screen. In the case of the image that has been captured from the front direction, for example, a point 34c at the center of both eyes for the head part, a point 35c at the center of the acromioclavicular joints 35a and 35b of both shoulders for the chest part, and a point 36c at the center of the left and right anterior superior iliac spines 36a and 36b in a part corresponding to the pelvis for the pelvis part can be set as the predetermined points identified in step S6.

FIG. 4 is a diagram illustrating an example of a display screen according to an embodiment of the present invention. A captured image of the client's body from the lateral side direction is displayed on the display screen. In the case of the image that has been captured from the lateral side direction, for example, an occipital external protuberance 37c for the head part, a part 38c corresponding to near the fourth spinous process of thoracic vertebra to the fifth spinous process of thoracic vertebra for the chest part, and the second spinous process of vertebra 39b for the pelvis part can be set as the predetermined points.

In step S6, a point for identifying a positional deviation of the body site may be identified in the image that has been captured from either one of the front direction or the lateral side direction. However, a point for identifying the positional deviation of the body site may be identified in the images that have been captured from a plurality of directions such as the front direction and the lateral side direction. For example, with use of the image from the front direction illustrated in FIG. 3 and the image from the lateral side direction illustrated in FIG. 4, two predetermined points or one predetermined point of each body site is identified in a multilateral manner from two directions of the front direction and the lateral side direction. By associating the points identified from the two directions of the front direction and the lateral side direction with each other to assess the positional deviation of the body site, it becomes possible to grasp the positional deviation of each body site in the left-and-right direction and the front-and-rear direction more accurately than the case of assessing the deviation from either one of the directions.

In addition, with use of the images that have been captured from the rear direction and the top direction, in addition to the images that have been captured from the front direction and the lateral side direction, by identifying the points to be used for identifying the positional deviation of the body site from three directions or three-dimensionally with regard to the client's posture, it becomes possible to grasp the positional deviation of each body site in a more accurate manner and in more detail. Hereinafter, a description will be given, as an example, with use of the drawings, for a case where a predetermined point is identified in an image that has been captured from the rear direction, in addition to the images that have been captured from the front surface direction and the lateral side direction.

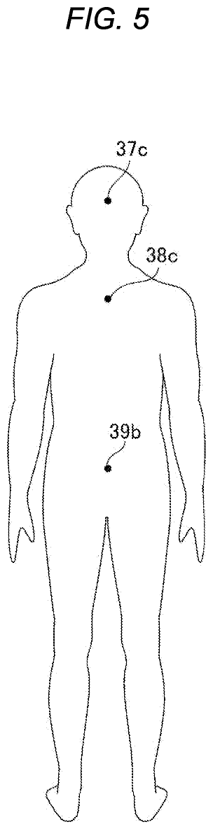

FIG. 5 is a diagram illustrating an example of a display screen according to an embodiment of the present invention. A captured image of a client's body from the rear direction is displayed on the display screen. In the case of the image that has been captured from the rear direction, for example, the occipital external protuberance 37c for the head part, the part 38c corresponding to near the fourth spinous process of thoracic vertebra and the fifth spinous process of thoracic vertebra for the chest part, and the second spinous process of vertebra 39b for the pelvis part can be set as the predetermined points.

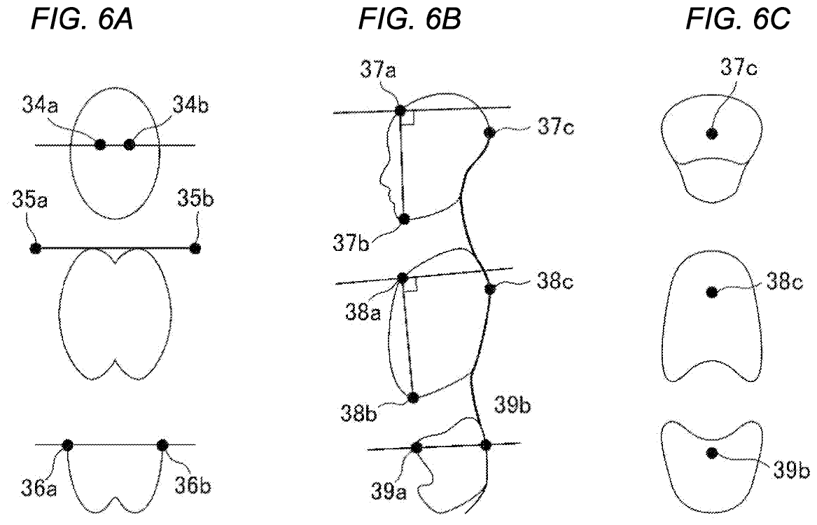

FIG. 6 is a diagram illustrating an example of a display screen according to an embodiment of the present invention. FIGS. 6A, 6B, and 6C illustrate schematic diagrams in a case where the predetermined points are identified with the head part, the chest part, and the pelvis part as body sites to be used as point identification targets respectively in the images that have been captured from the front direction, the lateral side direction, and the rear direction. By use of the predetermined points of the body site identified in the images that have been captured from these three directions, it becomes possible to grasp the positional deviation of the body site in a more accurate manner and in more detail than the case where the positional deviation of the body site is assessed from either one of the front direction or the lateral side direction or two directions of the front direction and the lateral side direction. For example, for the head part, by assessing the positional relationship between the centers 34a and 34b of both eyes that have been identified from the image in the front direction and the occipital external protuberance 37c that has been identified from the images in the lateral side direction and the rear direction, it becomes possible to grasp an inclination in the left-and-right direction of the occipital external protuberance 37c in a case where the front-and-rear direction of the body is used as an axis. In addition, for example, for the head part, by assessing the positional relationship between the glabella 37a and the chin tip 37b that have been identified from the image in the lateral side direction and the occipital external protuberance 37c that has been identified from the images in the lateral side direction and the rear direction, it becomes possible to grasp a deviation in the front-and-rear direction of the occipital external protuberance 37c in a case where the left-and-right direction of the body is used as an axis.

Further, for example, in addition to the images that have been captured from the front direction, the lateral side direction, and the rear direction, a point identified from an image that has been captured from a top direction, although not illustrated, may be combined to assess a positional deviation of the body site. In a case of configuring this manner, for the head part, by assessing the positional relationship between the glabella 37a that has been identified from the image in the top direction and the occipital external protuberance 37c that has been identified from the images in the lateral side direction and the rear direction, it is possible to grasp a deviation of the occipital external protuberance 37c in the left-and-right direction in a case where an up-and-down direction of the body is used as an axis. In addition, instead of using the image from the top direction, with use of an image that has been captured by a depth camera with a depth sensor, by measuring a depth from the point identified from the image in the front direction to the point identified from the image in the rear direction, it is possible to grasp the positional deviation of the body site in a similar manner to the case of using the image from the top direction. In this manner, by assessing the positional relationship between the points identified in the images that have been captured from the plurality of directions or the points identified by the depth sensor in addition to the images that have been captured from the plurality of directions, it becomes possible to grasp the orientation of each body site and the positional deviation of the body site in an accurate manner and in detail.

In identifying the point of the body site in step S6, it is possible to identify the point by performing a touch operation on the touch panel with a finger. However, for example, the user may identify the point by performing a touch operation on the touch panel with a stylus, or the user may identify the point by moving a cursor to a desired point on the image by operating the input unit 22. In addition, separately from the method for identifying points on the body site in accordance with an operation of the user, a method for automatically identifying two predetermined points of the body site from the image data may be adopted in accordance with a predetermined computer program or by a process performed by AI.

Next, a positional deviation of the body site is identified, based on the point identified for each body site (step S7). For example, it is possible to identify the positional deviation of the body site with a parameter such as an angle formed by a line segment connecting two points identified for different body sites as identified in step S6 and a straight line perpendicular to a horizontal plane in the image, or a unit vector starting from either one of the points and ending at the other, or the like.

The orientation of each body site and the positional deviation between the body sites identified in steps S5 and S7 are stored in the storage unit 13 (step S8).

On the display screen 19a of the computer apparatus 1 possessed by the user, information regarding the orientation of the body site identified in step S5 is displayed (step S9). For example, the parameter itself identified in step S5 may be displayed on the display screen, so that the user can objectively grasp the orientation of the body site. Further, as illustrated in FIG. 4, information regarding the orientation of the body site identified in step S5 may be displayed with use of an object such as an arrow starting from the point identified in step S6. In the case of the head part 31, the orientation of the body site is displayed by an arrow 37d, and in the case of the chest part 32, the orientation of the body site is displayed by an arrow 38d. In the case of the pelvis part 33, the orientation of the body site is displayed by an arrow 39d. In this manner, the information regarding the orientation of the body site is displayed with use of the arrow starting from the point for grasping the positional deviation of the body site. Thus, the user is able to easily grasp the positional deviation and the orientation of the body site. In addition, as long as it facilitates visually grasping the deviation and the orientation of the body site, a line connecting the points identified in step S4 and/or step S6, a block representing the body site, or the like may be used for displaying the information regarding the orientation of the body site, instead of an arrow.

By performing the processes of steps S1 to S9, the posture assessment process is terminated.

The user is able to grasp the conditions of the muscles, based on the posture state displayed on the display screen 19a, in step S9. For example, in a case where the chest part 32 is directed downward as being located forward, and the pelvis part 33 is inclined upward as being located forward, it can be understood that muscles in the vicinity of the chest part 32 and the pelvis part 33 on a front side of the body are in a hypertonia (contracted) condition, and muscles in the vicinity of the chest part 32 and the pelvis part 33 on a rear side of the body are in a hypotonic (relaxed) condition.

In identifying the position and orientation of the predetermined point of the body site in step S6, it is possible to identify the point of each body site in accordance with an operation of the user or a process of a computer program or AI, with use of images of the client's posture that have been captured from various directions.

Note that, here, the position and the orientation of the predetermined point of the body site are identified, based on the image. However, a motion capture sensor may be directly attached to a predetermined point of a body site so as to identify the position and the orientation of the predetermined point of the body site in a space. The client lowers both arms, stands on both legs in a direction perpendicular to a horizontal plane, and the position and the orientation of a predetermined point are measured in a stationary state. By use of the motion capture sensor, steps S1 to S4 can be omitted, and the processes from steps S5 to S9 are performed. Here, the predetermined points to which the motion capture sensors are attached are preferably points aligned on a straight line, in a case of a person in a normal posture. Note that any type such as an optical type, an inertial sensor type, a mechanical type, or a magnetic type may be used as the motion capture sensor.

In particular, in a case where the motion capture sensor is directly attached to a predetermined point of each body site to assess the posture, it is sufficient if one point with which it is possible to measure the orientation (including an inclination of the body site) and the positional deviation of the body site is identified for one body site to be a point identification target. Thus, it is possible to identify the predetermined point of the body site easily. Note that any type such as an optical type, an inertial sensor type, or a magnetic type may be used as the motion capture sensor. In a case where an optical sensor is used, a reflective marker is attached to a predetermined point. In a case where the inertial sensor type is used, a gyro sensor is attached to the predetermined point. In addition, in a case where the magnetic type is used, a magnetic sensor is attached to the predetermined point.

The information regarding the position and the orientation of the predetermined point obtained by the motion capture sensor is transmitted to the computer apparatus 1 on wireless communication, and the processes of steps S5 to S9 are performed.

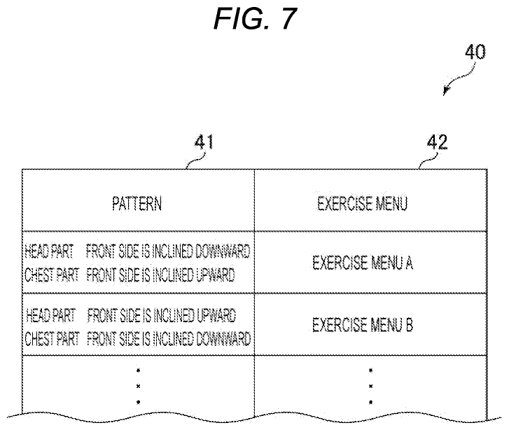

In the present invention, by the way, it is also possible to identify an appropriate exercise menu based on the parameters identified in steps S5 and S7, and to recommend such an appropriate exercise menu to the user. FIG. 7 is a diagram illustrating an exercise menu table according to an embodiment of the present invention. In an exercise menu table 40, an exercise menu 42 appropriate for a pattern 41 of the orientation of each body site is set in association with the pattern 41. Then, with reference to the exercise menu table 40, it is possible to identify an appropriate exercise menu 42 in accordance with which orientation pattern the parameter for each body site identified in step S5 corresponds to, and to display the identified exercise menu on the display screen 19a of the computer apparatus 1.

Here, examples of the orientation pattern of each body site include a combination of parameters of the orientations of the head part and the chest part, a combination of parameters of the orientations of the chest part and the pelvis part, and a combination of parameters of the orientations of the head part, the chest part, and the pelvis part. Therefore, for example, it is possible to identify different exercise menus between a case where the head part is inclined downward as being located forward of the body and the chest part is inclined upward as being located forward of the body and a case where the head part is inclined upward, and the chest part is inclined downward as being located forward of the body.

Note that, in the exercise menu table 40, an appropriate exercise menu may be set for the pattern in association with a positional deviation pattern of the body site. Then, with reference to the exercise menu table 40, it is possible to identify an appropriate exercise menu in accordance with which pattern the positional deviation for each body site identified in step S7 corresponds to, and to display the identified exercise menu on the display screen 19a of the computer apparatus 1.

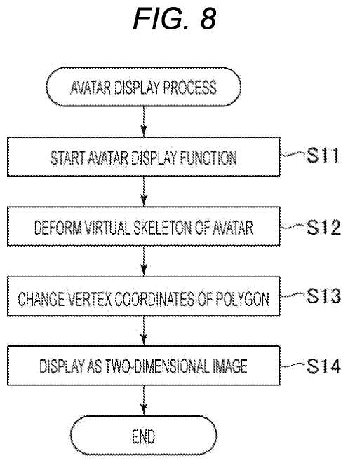

In the first embodiment of the present invention, it is also possible to display an avatar reflecting the client's posture on the display screen, based on the parameters identified in steps S5 and S7. FIG. 8 is a diagram illustrating an avatar display process according to an embodiment of the present invention. First, the user activates a dedicated application on the computer apparatus 1, and selects a start button of an avatar display function, and then the avatar display function is started (step S11).

A virtual skeleton is set in an avatar displayed on the display screen 19a by the avatar display function, and it is possible to cause the avatar to make a motion by moving the virtual skeleton that is movable. A plurality of types of avatars such as a male avatar and a female avatar are provided, and the user is able to appropriately select a desired avatar from these avatars. In addition, a virtual skeleton serving as a reference in an ideal posture is set in the avatar. When the avatar display function is started in step S11, the virtual skeleton of the avatar is deformed, based on the parameters specified in steps S5 and S7 (step S12).

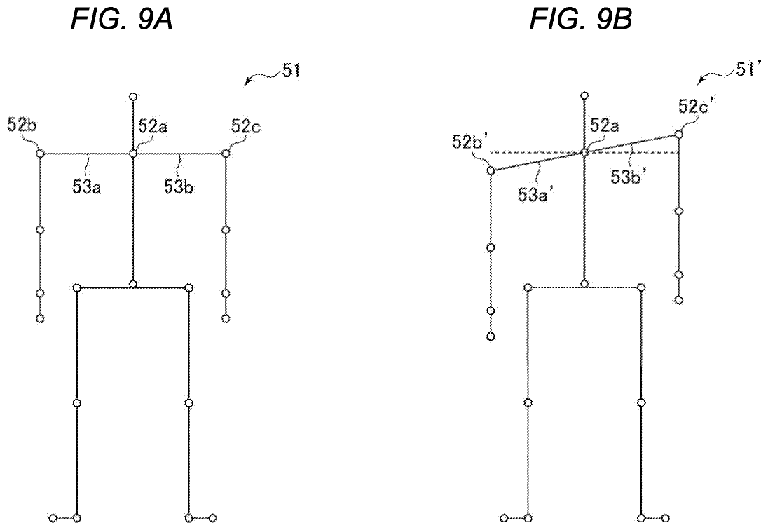

FIG. 9 is a diagram illustrating a virtual skeleton model according to an embodiment of the present invention. A virtual skeleton model 51 includes, for example, a plurality of virtual joints 52 (indicated by circles in FIG. 9) provided on movable parts, for example, a shoulder, an elbow, and a wrist, and a virtual skeleton 53 (indicated by straight lines in FIG. 9), which corresponds to an upper arm, a lower arm, a hand, and the like, and which has a linear shape for coupling the respective virtual joints 52. The deformation of the virtual skeleton in step S12 is made as follows.

For example, in a case where the orientation of the chest part is reflected on the virtual skeleton model 51, which serves as a reference and which is illustrated in FIG. 9A, the positions of virtual joints 52b and 52c are moved in accordance with the parameter corresponding to step S5, while the position of a virtual joint 52a is fixed, and the virtual joint 52a and the virtual joints 52b and 52c on the both sides are maintained to be aligned on a straight line. For example, the virtual joint 52b is moved downward, and the virtual joint 52c is moved upward. As a result, virtual skeletons 53a and 53b also move.

FIG. 9B illustrates a virtual skeleton model 51′ after deformation. The virtual skeleton 53 can be defined by the coordinates of the virtual joints at its both ends, and thus a virtual skeleton 53a′ after deformation can be defined by the coordinates of virtual joints 52a′ and 52b′ after deformation. A virtual skeleton 53b′ after deformation can be defined by the coordinates of the virtual joints 52a′ and 52c′ after deformation. A similar process can be performed for the other virtual joints 52 and the other virtual skeletons 53.

Vertex coordinates of a plurality of polygons are associated with the virtual skeleton 53 in order to visualize the avatar. When the virtual skeleton 53 is deformed in step S12, the vertex coordinates of the associated polygons are also changed in accordance with the deformation (step S13). By rendering model data of the avatar including the polygons, the vertex coordinates of which have been changed, it is possible to display the avatar as a two-dimensional image or a three-dimensional image (step S14). By performing the steps S11 to S14, the avatar display process ends.

Note that in displaying the avatar in steps S11 to S14, it is also possible to provide a motion for the avatar and display the avatar. In a motion program for providing a motion for the avatar, by determining an angle of a corner formed at each virtual joint 52 at the time of motion start and after the motion end (for example, an angle of a corner of the shoulder part formed by three joint points of the elbow, the shoulder, and the neck part) and an angular velocity at the time of the motion for such an angle, and changing the angle formed at the virtual joint 52 as the time elapses, it is possible to provide a predetermined motion.

### Second Embodiment

Next, an outline of a second embodiment of the present invention will be described. Hereinafter, as the second embodiment, a description will be given, as an example, for a system for assessing a client's posture to be implemented on a computer apparatus and a server apparatus connectable with the computer apparatus through communication.

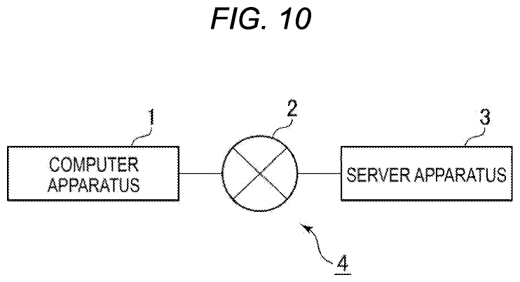

FIG. 10 is a block diagram illustrating a configuration of a posture assessment system according to an embodiment of the present invention. As illustrated in the drawing, a system 4 in the present embodiment includes a computer apparatus 1 operated by a user, a communication network 2, and a server apparatus 3. The computer apparatus 1 is connected with the server apparatus 3 through the communication network 2. The server apparatus 3 does not have to be always connected with the computer apparatus 1, and it is sufficient if the server apparatus 3 is connectable with the computer apparatus 1 as necessary.

Regarding a specific configuration of the computer apparatus 1, the contents that have been described in the first embodiment can be adopted within a necessary range. In addition, for example, the server apparatus 3 includes at least a control unit, a RAM, a storage unit, and a communication interface, and can be configured such that these units are connected with one another through an internal bus. The control unit includes a CPU and a ROM, and includes an internal timer that counts time. The control unit executes a program stored in the storage unit, and controls the server apparatus 3. The RAM is a work area of the control unit. The storage unit is a storage area for storing programs and data. The control unit reads the program and data from the RAM, and performs a process of executing the program, based on information or the like that has been received from the computer apparatus 1.

The communication interface is connectable with the communication network 2 wirelessly or by wire, and is capable of transmitting and receiving data through the communication network 2. Data that have been received through the communication network 2 are loaded onto the RAM, for example, and an arithmetic process is performed by the control unit.

In the posture assessment system according to the second embodiment, processes similar to the posture assessment process illustrated in FIG. 2 and the avatar display process illustrated in FIG. 8 are performed on either the computer apparatus 1 or the server apparatus 3.

Hereinafter, a posture assessment process in the posture assessment system will be described. When the user activates a dedicated application installed in the computer apparatus 1, and selects a start button of a camera function, the camera function is started. The user uses the computer apparatus 1 to image a part or an entirety of the client's body, for example, from a front direction or from a lateral side direction. An image of the client is captured in a state of standing in a direction perpendicular to a horizontal plane.

Note that, here, the image of the client is captured with the camera function. However, image data of an image that has been captured by another computer apparatus or the like may be taken into and used on the computer apparatus.

Next, the captured image of the client is displayed on the display screen 19a. Then, the user operates the computer apparatus 1, and transmits image data of the image of the client that has been captured to the server apparatus 3. The server apparatus 3 identifies at least two points for each body site, based on the image data of the image of the client that has been received from the computer apparatus 1. Examples of the body site to be a point identification target include the head part, the chest part, and the pelvis part. These two points are used for identifying the orientation of the body site, and which part of the body should be set as two predetermined points is preferably determined beforehand for each body site. In addition, even in the same body site, the two predetermined points are preferably different between a case where the image has been captured from the front direction and a case where the image has been captured from the lateral side direction.

Next, in addition to the two points identified as described above, the server apparatus 3 further identifies at least one point for each body site. Examples of the body site to be a point identification target include the head part, the chest part, and the pelvis part, but another body site may be included as the point identification target. Such one point is used for identifying a positional deviation of the body site, and which part of the body should be set as one predetermined point is preferably determined beforehand for each body site. Further, even in the same body site, one predetermined point is preferably different between a case where the image has been captured from the front direction and a case where the image has been captured from the lateral side direction. Further, the point identified in this timing may include the point that has been identified for identifying the orientation of a body site. That is, the point identified for identifying the orientation of the body site and the point identified for identifying the positional deviation of the body site may be the same with each other, or may be different from each other. In addition, these predetermined points of the body site to be identified are preferably points aligned on a straight line, in a case of a person in a normal posture.

The server apparatus 3 identifies the orientation of each body site with a parameter based on the two points that have been identified for each body site. In addition, the server apparatus 3 identifies the positional deviation of the body site with a parameter based on the point that has been identified for each body site. In the server apparatus 3, the parameter for the orientation of each body site and the parameter for the positional deviation of the body site are stored in the storage unit.

The computer apparatus 1 receives the parameter for the orientation of the body site and the parameter for the positional deviation of the body site. On the display screen 19a of the computer apparatus 1, information regarding the orientation of the body site and the positional deviation of the body site is displayed, based on the parameters that have been received. Specifically, video images similar to those illustrated in FIGS. 3 and 4 are displayed on the display screen 19a of the computer apparatus 1.

The posture assessment system in the second embodiment is preferably configured to display an avatar reflecting the client's posture on the display screen, based on the parameters identified in steps S5 and S7. When receiving the image data of the image of the client that has been transmitted from the computer apparatus 1 in accordance with an operation of the user, and identifying the parameter for the orientation of each body site and the parameter for the positional deviation of the body site, the server apparatus 3 creates image data of the avatar in order to display the avatar reflecting the client's posture on the display screen 19a of the computer apparatus 1.

Note that also in the second embodiment, steps S1 to S4 can be omitted by use of a motion capture sensor in the posture assessment process, and the processes from steps S5 to S9 can be performed.

When the creation of the image data of the avatar is started, the server apparatus 3 performs a process of deforming a virtual skeleton of the avatar, based on the parameter for the orientation of each body part and the parameter for the positional deviation of the body site. In the process of deforming the virtual skeleton, a process similar to step S12 described above can be performed.

Vertex coordinates of a plurality of polygons are associated with the virtual skeleton in order to visualize the avatar. The vertex coordinates of the associated polygons are also changed in accordance with the deformation of the virtual skeleton. The two-dimensional image data or the three-dimensional image data of the avatar obtained by rendering the avatar model data including the polygon, the vertex coordinates of which have been changed, is transmitted from the server apparatus 3 to the computer apparatus 1. The computer apparatus 1 receives the two-dimensional image data or the three-dimensional image data of the avatar, and displays the avatar on the display screen. Also for the avatar, the virtual skeleton of which has been deformed, the server apparatus 3 executes a motion program to display the avatar, for which the computer apparatus 1 has provided the motion.

In the computer apparatus 1, with use of the avatar display function, it is possible to switch between the image of the avatar and the image of the client, and to display an image in which the image of the virtual skeleton of the avatar is superimposed on the image of the client, on the display screen 19a. With this configuration, the client is able to grasp the orientation of the body site and the positional deviation of the body site more accurately and visually, based on the captured image of the client's posture, and is then able to do an exercise in a proper form. In addition, with this configuration, for example, when an instruction or advice is received from a trainer or another third party via an online service using the Internet, the image of the avatar is displayed on the display screen of a computer apparatus on the other side, so that the avatar image can be used for protecting the privacy of the client.

In the posture assessment system in the second embodiment, the information regarding the orientation and the positional deviation of the body site received by the computer apparatus 1 from the server apparatus 3 may be received not only as the virtual skeleton model indicating the proper posture but also as a posture score. In this configuration, it is possible to calculate the posture score by quantifying the parameters of the position and the orientation of the body site, and to display the posture score on the display screen for each client. For example, as the orientation of the body site is closer to normal, the posture score becomes higher. As the orientation of the body site is separated from the normal state, the posture score becomes lower.

In the second embodiment, the description has been given, as an example, for the system implemented by the computer apparatus and the server apparatus connectable with the computer apparatus through communication. However, a portable computer apparatus can be used instead of the server apparatus. That is, the configuration is also applicable to a peer-to-peer system including a computer apparatus such as a smartphone and a similar computer apparatus such as a smartphone.

### Third Embodiment

An outline of a third embodiment of the present invention will be described. According to a method in the third embodiment, for example, an image of the client's posture can be captured by a trainer or a client, so that the client's posture can be grasped, based on the captured image. In the third embodiment, the client's posture can be grasped without use of the computer apparatus.

A posture assessment method according to an embodiment of the present invention will be described. The trainer or the client prints out, on paper or the like, an image of a part or an entirety of the client's body that has been captured from, for example, a front direction or a lateral side direction. The trainer or the client visually recognizes the printed image, and identifies at least two points for identifying the orientation of the body site from among body sites. Examples of the body site to be two point identification targets include the head part, the chest part, and the pelvis part, but another body site may be included as a point identification target. The trainer or the client writes a mark at the two points that have been identified in the image.

The trainer or the client identifies one point for grasping a positional deviation of a body site from among body sites, separately from the two points that have been identified above. Examples of the body site to be a point identification target include the head part, the chest part, and the pelvis part, but another body site may be included as the point identification target. In the image that has been captured from the front direction, the points to be identified in the head part, the chest part, and the pelvis part are preferably points aligned on a straight line, in a case of a person in a normal posture. Similarly, in the image that has been captured from the lateral side direction, the points to be identified in the head part, the chest part, and the pelvis part are preferably points aligned on a straight line, in a case of a person in a normal posture. The trainer or the client writes a mark at the point that has been identified in the image.

Next, the trainer or the client then writes, in the image, the orientation of the body site to be obtained from the two identified points in the image. For example, the orientation of the body site is written as represented by an arrow. Here, it is possible to set a start point of the arrow so as to grasp the positional deviation of the body site. For example, as in the case where the glabella and the chin tip are identified for the head part, in a case where two points aligned to be perpendicular to the horizontal plane are identified in a normal posture, it is possible to set an arrow extending in a direction perpendicular to a line segment connecting the two points. For example, as in the case where the anterior superior iliac spine and the second spinous process of vertebra are identified for the pelvis part, in a case where two points aligned to be parallel to the horizontal plane are identified in a normal posture, it is possible to set an arrow extending in a direction parallel to a line segment connecting the two points.

### Fourth Embodiment

Next, an outline of a fourth embodiment of the present invention will be described. The fourth embodiment may be implemented as a computer apparatus similarly to the first embodiment, or may be implemented as a system including a computer apparatus and a server apparatus connectable with the computer apparatus through communication, in a similar manner to the second embodiment. The process to be described below may be performed by either the computer apparatus 1 or the server apparatus 3, except the process that can be performed only on the computer apparatus 1.

In the fourth embodiment, it is possible to display the avatar on the display screen 19a, while changing the orientation of the body site and the positional deviation of the body site. First, the user activates a dedicated application on the computer apparatus 1, and selects a start button of an avatar display function, and then the avatar display function is started.

A virtual skeleton is set in the avatar, so that it is possible to cause the avatar to make a motion by moving the virtual skeleton that is movable. A reference virtual skeleton in an ideal posture that serves as a reference is set in the avatar, so it is possible to deform the virtual skeleton that serves as a reference. The deformation of the virtual skeleton is made in accordance with an operation of the user on the computer apparatus 1. More specifically, by changing the orientation of the front-and-rear direction or the left-and-right direction of an entirety or a part of the virtual skeleton of any one of the head part, the chest part, and the pelvis part, it is possible to deform the virtual skeleton. In addition, by shifting the position of an entirety or a part of the virtual skeleton of any one of the head part, the chest part, and the pelvis part in the front-and-rear direction or the left-and-right direction, it is possible to deform the virtual skeleton.

In FIG. 9, for example, in a case where the orientation of the chest part is reflected on the virtual skeleton that serves as a reference, the positions of virtual joints 52b and 52c are moved in accordance with an input made by the user, while the position of the virtual joint 52a is fixed, and the virtual joint 52a and the virtual joints 52b and 52c on the both sides are maintained to be aligned on a straight line. For example, the virtual joint 52b is moved downward, and the virtual joint 52c is moved upward. The virtual skeleton 53 can be defined by the coordinates of the virtual joints 52 at its both ends. Thus, the virtual skeleton 53a′ after deformation can be defined by the coordinates of the virtual joints 52a′ and 52b′ after deformation. A virtual skeleton 53b′ after deformation can be defined by the coordinates of the virtual joints 52a′ and 52c′ after deformation.

When the virtual skeleton is deformed, the vertex coordinates of the associated polygons are also changed in accordance with the deformation. By rendering the model data of the avatar including the polygons, it is possible to display the avatar as a two-dimensional image. In addition, as described in the first embodiment, with use of the motion program, it is possible to provide a motion for the avatar. In this case, in accordance with an operation of the user, it is possible to cause the avatar to make a predetermined motion, while changing the orientation and the positional deviation of the body site of the avatar.

### Fifth Embodiment

An outline of a fifth embodiment of the present invention will be described. According to a posture assessment system in the fifth embodiment, for example, while a trainer and a client are making a real-time online session through a communication network, it is possible to provide an environment in which information regarding the orientation and the positional deviation of the body site, the orientation of the body site, and the like is shared with the trainer, based on an image that has been captured by the client itself with a smartphone or the like, so that an instruction for an appropriate exercise menu can be received from the trainer remotely.

The system according to the present embodiment includes a first apparatus operated by a user, a communication network, and a second apparatus connectable with the first apparatus through communication. The first apparatus is connected with the second apparatus through the communication network.

Regarding a specific configuration of the first apparatus and/or the second apparatus, the contents related to the computer apparatus described in the first embodiment can be adopted within a necessary range. In addition, for example, the second apparatus includes at least a control unit, a RAM, a storage unit, and a communication interface, and can be configured such that these units are connected with one another through an internal bus. The control unit includes a CPU and a ROM, and includes an internal timer that counts time. The control unit executes a program stored in the storage unit, and controls the second apparatus. The RAM is a work area of the control unit. The storage unit is a storage area for storing programs and data. The control unit reads the program and data from the RAM, and performs a process of executing the program, based on information or the like that has been received from the first apparatus.

The communication interface is connectable with the communication network wirelessly or by wire, and is capable of transmitting and receiving data through the communication network. Data that have been received through the communication network are loaded onto the RAM, for example, and an arithmetic process is performed by the control unit.

In the posture assessment system according to the fifth embodiment, processes similar to the posture assessment process illustrated in FIG. 2 and the avatar display process illustrated in FIG. 8 are performed in either the first apparatus or the second apparatus.

Hereinafter, a posture assessment process in the posture assessment system will be described. First, an online session using the communication network is started between the first apparatus and the second apparatus by an operation on the first apparatus by the client or an operation on the second apparatus by the trainer. The online session may be directly made between the first apparatus and the second apparatus by a dedicated application installed in the first apparatus and the second apparatus, or may be made via a server on a cloud network with use of a conventionally known communication application or social networking service. When the online session is started, the client uses the camera function of the first apparatus to capture an image of a part or an entirety of the client's body, for example, from the front direction or the lateral side direction.

Note that, here, the image of the client is captured with the camera function. However, image data of an image that has been captured by another computer apparatus or the like may be taken into and used on the client's own computer apparatus, or the posture of the client may be intermittently captured by the camera function to be input to the first apparatus in real time. In addition, transmission and reception may be intermittently conducted between the first apparatus and the second apparatus via streaming or live distribution with use of a file transfer system such as Peer to Peer.

Next, the captured image of the client is displayed on the display screen of the first apparatus and/or the second apparatus. In a case of identifying a point for each body site on the first apparatus carried by the client, as soon as an image of the client's posture is captured, the first apparatus performs the posture assessment process based on the captured image so as to identify a point for identifying the orientation and the positional deviation of the body site from among the body sites. Examples of the body site to be a point identification target include the head part, the chest part, and the pelvis part, but another body site may be included as the point identification target. Then, the orientation of the positional deviation of the body site is identified by parameters, based on the point that has been identified for each body site.

Next, in the first apparatus, a parameter for the orientation of each body site and a parameter for the positional deviation of the body site are stored in the storage unit. Then, on the display screen of the first apparatus, information regarding the orientation of the body site and the positional deviation of the body site is displayed, based on the parameters that have been stored.

In addition, the user operates the first apparatus, and transmits image data of the captured image of the client to the second apparatus. Instead of the image data of the captured image of the client, the parameter for the orientation of the body site and the parameter for the positional deviation of the body site may be transmitted. The second apparatus receives the image data of the image of the client, or the parameter for the orientation of the body site and the parameter for the positional deviation of the body site. Then, on a display screen of the second apparatus, information regarding the orientation of the body site and the positional deviation of the body site is displayed, based on the image data of the image of the client that has been received or the parameters that have been received.

The posture assessment system in the fifth embodiment is preferably configured to display an avatar reflecting the client's posture on the display screen, based on the parameters identified in steps S5 and S7. With this configuration, for example, when the client transmits the image to the computer apparatus possessed by the trainer via an online session with the trainer, in a case where the client does not desire to transmit the captured image of the client to the trainer directly, the image of the avatar is displayed on the display screen of the computer apparatus on the trainer side, so that the avatar image can be used for protecting the privacy of the client. Further, for example, by enabling the image in which the image of the virtual skeleton of the avatar is superimposed on the image of the client to be displayed, the avatar of the virtual skeleton of the ideal posture is superimposed on the captured image of the client's posture, so that the orientation and the positional deviation of the body site can be grasped more accurately and visually to encourage the user to do exercise in a proper form.

In an embodiment of the present invention, in a case where the orientation and the positional deviation of the body site is identified from one point that has been identified, as long as the orientation and the positional deviation of the body site are identifiable from one point that has been identified, any of the body sites may be used for identifying one predetermined point.

In an embodiment of the present invention, the posture of an assessed person is grasped by identifying the orientation and the positional deviation of the body site at an identified point of the body site, but the present invention is not limited to this. The posture of the assessed person may be grasped by identifying a “position” of a predetermined point identified in the body site and a “orientation” (inclination) of the body site at the “position”.

In an embodiment of the present invention, the description has been given, as an example, for a case where the parameter for the orientation of each body site or the parameter for the positional deviation of the body site is stored in the storage unit of the computer apparatus. However, a storage area for storing various types of data related to the posture assessment identified in the posture assessment system according to the present invention is not limited to a storage unit in a computer apparatus. The computer apparatus may be configured to be connected with a communication network to store data in a cloud storage on an external cloud network.

### REFERENCE SIGNS LIST

1 COMPUTER APPARATUS
2 COMMUNICATION NETWORK
3 SERVER APPARATUS
4 SYSTEM
11 CONTROL UNIT
12 RAM
13 STORAGE UNIT
14 SOUND PROCESSING UNIT
15 SOUND OUTPUT DEVICE
16 SENSOR UNIT
17 FRAME MEMORY
18 GRAPHICS PROCESSING UNIT
19 DISPLAY UNIT
20 COMMUNICATION INTERFACE
21 INTERFACE UNIT
22 INPUT UNIT
23 CAMERA UNIT

1-27. (canceled)

28. A posture assessment apparatus comprising:
an orientation identifier configured to identify an orientation of the body site, based on at least two points of a body site of an assessed person, and
an orientation displayer configured to display information regarding the orientation that has been identified in association with at least one point of a body site of the assessed person, wherein
the point that has been associated with the information regarding the orientation is different from any of the points of identifying the orientation of the body site.

29. The posture assessment apparatus according to claim 28, wherein
the orientation identifier identifies an orientation that can be represented with use of a normal line of the line segment connecting the two points of identifying the orientation of the body site as the orientation of the body site.

30. The posture assessment apparatus according to claim 28, wherein
the points that have been associated with the information regarding the orientation of the body site are points to be aligned on a straight line, in a case of a person in a normal posture.

31. The posture assessment apparatus according to claim 28, wherein
the orientation identifier and/or the orientation displayer is performed based on images that have been captured from the front direction, the lateral side direction, and the rear direction or images that have been captured from the front direction, the lateral side direction, and the top direction;
the points that have been associated with the information regarding the orientation of the body site are different from any of the points of identifying the orientation of the body site; and
the points that have been associated with the information regarding the orientation of the body site are points to be aligned on a straight line, in a case of a person in a normal posture.

32. The posture assessment apparatus according to claim 28, wherein
the orientation identifier and/or the orientation displayer is performed based on images that have been captured from the front direction, the lateral side direction, and the rear direction or images that have been captured from the front direction, the lateral side direction, and the top direction.

33. The posture assessment apparatus according to claim 28, the apparatus further comprising:
a position identifier configured to identify the position of the plurality of body sites;
a virtual skeleton change part configured to change a virtual skeleton set in a virtual model in accordance with the orientation that has been identified by the orientation identifier and/or the position that has been identified by the position identifier; and
a virtual model displayer configured to render a virtual model in accordance with the virtual skeleton that has been changed, and configured to display the virtual model as a two-dimensional image or a three-dimensional image.

34. A posture assessment method to be performed on a computer apparatus, the posture assessment method comprising:
identifying an orientation of the body site, based on at least two points of a body site of an assessed person, and
displaying information regarding the orientation that has been identified in association with at least one point of a body site of the assessed person, wherein
the point that has been associated with the information regarding the orientation is different from any of the points of identifying the orientation of the body site.

35. The posture assessment method according to claim 34, wherein
the orientation of the body site is identified an orientation that can be represented with use of a normal line of the line segment connecting the two points of identifying the orientation of the body site as the orientation of the body site.

36. The posture assessment method according to claim 34, wherein
the points that have been associated with the information regarding the orientation of the body site are points to be aligned on a straight line, in a case of a person in a normal posture.

37. The posture assessment method according to claim 34, wherein
identifying and/or displaying is performed based on images that have been captured from the front direction, the lateral side direction, and the rear direction or images that have been captured from the front direction, the lateral side direction, and the top direction;
the points that have been associated with the information regarding the orientation of the body site are different from any of the points of identifying the orientation of the body site; and
the points that have been associated with the information regarding the orientation of the body site are points to be aligned on a straight line, in a case of a person in a normal posture.

38. The posture assessment method according to claim 34, wherein
identifying and/or displaying is performed based on images that have been captured from the front direction, the lateral side direction, and the rear direction or images that have been captured from the front direction, the lateral side direction, and the top direction.

39. The posture assessment method according to claim 34, the method further comprising:
identifying the position of the plurality of body sites;
changing a virtual skeleton set in a virtual model in accordance with the orientation that has been identified and/or the position that has been identified; and
rendering a virtual model in accordance with the virtual skeleton that has been changed, and displaying the virtual model as a two-dimensional image or a three-dimensional image.

40. A posture assessment system comprising:
a first apparatus and a second apparatus capable of conducting a communication connection with the first apparatus; and
an orientation identifier configured to identify an orientation of the body site, based on at least two points of a body site of an assessed person, and
an orientation displayer configured to display information regarding the orientation that has been identified in association with at least one point of a body site of the assessed person, wherein
the point that has been associated with the information regarding the orientation is different from any of the points of identifying the orientation of the body site.

41. The posture assessment system according to claim 40, wherein
the orientation identifier identifies an orientation that can be represented with use of a normal line of the line segment connecting the two points of identifying the orientation of the body site as the orientation of the body site.

42. The posture assessment system according to claim 40, wherein
the points that have been associated with the information regarding the orientation of the body site are points to be aligned on a straight line, in a case of a person in a normal posture.

43. The posture assessment system according to claim 40, wherein
the orientation identifier and/or the orientation displayer is performed based on images that have been captured from the front direction, the lateral side direction, and the rear direction or images that have been captured from the front direction, the lateral side direction, and the top direction;
the points that have been associated with the information regarding the orientation of the body site are different from any of the points of identifying the orientation of the body site; and
the points that have been associated with the information regarding the orientation of the body site are points to be aligned on a straight line, in a case of a person in a normal posture.

44. The posture assessment system according to claim 40, wherein
the orientation identifier and/or the orientation displayer is performed based on images that have been captured from the front direction, the lateral side direction, and the rear direction or images that have been captured from the front direction, the lateral side direction, and the top direction.

45. The posture assessment system according to claim 40, the system further comprising:
a position identifier configured to identify the position of the plurality of body sites;
a virtual skeleton change part configured to change a virtual skeleton set in a virtual model in accordance with the orientation that has been identified by the orientation identifier and/or the position that has been identified by the position identifier; and
a virtual model displayer configured to render a virtual model in accordance with the virtual skeleton that has been changed, and configured to display the virtual model as a two-dimensional image or a three-dimensional image.
